(19) United States
(12) Patent Application Publication
(10) Pub. No.: US 2026/0102943 A1
(43) Pub. Date: Apr. 16, 2026

(54) HYDRAULIC PRESSURE (HP)/SLUMP ALERTING TECHNIQUE

(71) Applicant: CiDRA Concrete Systems Inc., Wallingford, CT (US)

(72) Inventors: John A. BIESAK, Asbury Park, NJ (US); Christopher Donlon GATCOMB, Wallingford, CT (US); Douglas Harold LOOSE, Southington, CT (US); Chase Michael MANNING, Mystic, CT (US); David Vincent NEWTON, Madison, CT (US); Maxwell Martin SEIFERT, East Granby, CT (US)

(21) Appl. No.: 18/871,871

(22) PCT Filed: Jun. 26, 2023
(86) PCT No.: PCT/US2023/026208

§ 371 Date: Dec. 5, 2024

Related U.S. Application Data
(60) Provisional application No. 63/355,731, filed on Jun. 27, 2022.

(51) Int. Cl. B28C 5/42; B28C 7/02; G01N 33/38

(52) U.S. Cl. CPC B28C 5/422; B28C 7/026; G01N 33/383

(57) ABSTRACT

Apparatus includes a signal processor or processing module configured to receive signaling containing information about a hydraulic pressure of a hydraulic fluid used or required to rotate a drum of a concrete truck having concrete contained therein; and provide corresponding signaling containing information about a slump alert of the concrete contained in the drum, based upon the signaling received. The signal processor or processing module determines the slump alert based upon a measurement of the hydraulic pressure of the hydraulic fluid used or required to rotate the drum at a specific rotation speed. The apparatus includes a memory having a look-up table containing information about reference slumps for corresponding reference concretes that are in-specification and out-of-specification based upon the hydraulic pressure measured. The signal processor or processing module may be configured to index the look-up table based upon the signaling.

### CROSS-REFERENCE TO RELATED APPLICATIONS

This application claims benefit to provisional patent application Ser. Nos. 63/355,731 (712-002.470 (CCS-0219)), filed on 27 Jun. 2022, which is hereby incorporated by reference in its entirety.

This application relates to U.S. Pat. No. 10,156,547 (WFMB no. 712-002.365-1-1 (CCS-0075, 67, 104)), which corresponds to PCT/US2012/060822, filed 18 Oct. 2012, claiming benefit to provisional patent application Ser. Nos. 61/548,549 and Ser. No. 61/548,563, both filed 18 Oct. 2011, which are all incorporated by reference in their entirety.

This application relates to patent application Ser. No. 16/638,258 (WFMB no. 712-002.451-1-1 (CCS-0143)), which corresponds to PCT/US2018/047429, filed 22 Aug. 2018, claiming benefit to provisional patent application Ser. Nos. 62/548,638 and 62/548,699, both filed on 22 Aug. 2017, which are all incorporated by reference in their entirety.

This application relates to patent application Ser. No. 16/638,237 (WFMB no. 712-002.452-1-1 (CCS-0200)), which corresponds to PCT/US2018/047479, filed 22 Aug. 2018, claiming benefit to provisional patent application Ser. Nos. 62/548,699 and 62/548,712, both filed on 22 Aug. 2017; which are all incorporated by reference in their entirety.

This application relates to patent application Ser. No. 16/638,237 (WFMB no. 712-002.467-1-1 (CCS-0216)), which corresponds to PCT/US2021/046211, filed 17 Aug. 2021, claiming benefit to provisional patent application Ser. Nos. 63/066,431, filed on 17 Aug. 2020; which are all incorporated by reference in their entirety.

The aforementioned applications were all assigned to the assignee of the present application, which builds on this family of technology.

### BACKGROUND OF THE INVENTION

### 1. Field of Invention

This invention relates to a technique for providing a slump alert for concrete in a rotating container or drum of a concrete truck.

### 2. Description of Related Art

The assignee of the instant patent application has developed so-called SMARThatch technology that provides concrete quality information to ready mix and precast producers. By way of example, this information may include air percent by volume, concrete temperature, volume returning from a job, drum rotation speed and direction, water added to load and drum motor hydraulic pressure. For examples, see that disclosed in the Assignee's aforementioned related technologies set forth above, which are all incorporated by reference.

The ‘slump’ of concrete refers to the workability or consistency of fresh concrete before it sets—the higher the slump, the more fluid the concrete is. Slump is used as a reference of how easy the concrete is to push, mold, and smooth, and a concrete's “Slump Rating” indicates what construction application the concrete is good for. The term “Slump” for concrete consistency got its name because it is traditionally measured by testing how much a pile of concrete slumps down when left to stand. The test is done by filling a cone with a sample of the concrete, removing the cone, and measuring how far the concrete has slumped. The number of inches the standard cone of concrete goes down (slumps down) from the top of the cone, after the cone is removed, is the measure of the slump.

The traditional method of measuring a concrete's slump is by using a “slump cone” which is a 12-inch tall truncated cone that is open at the top and bottom. The top is 4-inches wide and the bottom is 8 inches wide. After complete mixing of the concrete, the slump cone is used to measure the concrete's slump as follows:
1. Place the cone with the small side up on a smooth, nonabsorbent surface. Secure the cone so it doesn't move.
2. Fill the slump cone to ⅓ of its height with the prepared concrete.
3. Tamp down with 25 strokes of a ¾-inch steel rod, moving the rod around across the sample. An easy way to do this is to hold the rod at an angle to match the side of the cone. Start around the edges and move toward the middle.
4. Scoop in more concrete, filling the cone to the ⅔ mark, and tamp it with another 25 strokes. The rod should also go into the first layer about 1 inch.
5. Add more concrete to fill it to the top so it's slightly overflowing with one last layer and tamp it with a final 25 strokes. The rod should go into the second layer but not go through it completely.
6. Scrape off the excess concrete with the rod to create a smooth top.
7. Measure from the top of the cone to the base.
8. Pull the slump cone up off of the concrete. Pull the cone straight up quickly, being careful not to jerk it or turn it at an angle.
9. Measure how far the wet concrete sinks, or slumps down, by measuring from the top of the pile to the ground.
10. Subtract this measurement from the original height of the slump cone. The distance slumped down is called the slump level.

One problem with the known techniques is that many loads of concrete are lost each year because the slump is out-of-specification, meaning it can be too wet or too dry.

### SUMMARY OF THE INVENTION

The present invention builds on the assignee's so-called SMARThatch technology.

In general, by way of example, the assignee's so-called SMARThatch technology may be used to alert the concrete producer of these extreme situations in time to adjust the load and save it from rejection, according to some embodiments of the present invention. This is a critical capability/information for quality control and operations. In particular, drum motor hydraulic pressure can be used for such a slump correlation.

In particular, in a rotating drum ready-mix concrete truck, drum motor hydraulic pressure information, when utilized correctly, can also be an indicator of concrete slump/consistency of concrete. The hydraulic pressure required to rotate the ready-mix truck drum of a specific type of ready-mix truck will vary based on a number of factors, including the speed of drum rotation, the weight/amount of wet concrete in the drum, the mix design of the wet concrete, and the consistency (slump) of the wet concrete. Wetter concrete (higher slump concrete) will take less hydraulic pressure to turn the drum, and dryer, less workable concrete, will take more hydraulic pressure to turn the drum. The mix design of a concrete is understood to be the type of ingredients and the proportions thereof used to create the concrete that meets the technical specifications for a particular construction project.

Based upon this understanding, the present invention is designed to create alerts of potentially out-of-specification slump of concrete in the rotation drum of a ready-mix concrete truck by utilization of the measurement of the hydraulic pressure required to rotate the drum at a specified rotation speed.

The key to effective hydraulic pressure/slump alerts is the criteria built around how and when the hydraulic pressure measurement is made for purposes of providing alerts of in and out of specification slump. A reference look-up table is created that contains the following information for providing indications indicative of in and out of specification slump based on hydraulic pressure:
grouping of trucks (type of truck),
a mix design,
size of concrete load,
drum rotation speed, and
hydraulic pressure threshold limits-corresponding to in and out of specification slump.

By way of example, below is a slump alert table, as follows:

XYZ HP/Slump Alert Table
Table can get more specific based on QC feedback and
testing, See #9898/1234C5 mix design example

| Sample table | Mix Design Group/ | HP/Slump Wet | HP/Dry Dry |
|---|---|---|---|
| XYZ Trucks | by target slump | Alert | Alert |
| all trucks | a | <### | >#### |
| all trucks | b | <### | >#### |
| all trucks | c | <### | >#### |
| all trucks | d | <### | >#### |
| #9898 | 1234C5 | <### | >#### |

The time that the measurement is taken (truck status) can also be important. For example: a specific period of time after initial complete mixing of the concrete; and/or during transit for the ready mix truck to a delivery location; and/or upon arrival of a ready mix truck at a delivery location prior to pouring the concrete.

Alerts will include information such as truck number, date & time, thresholds, hydraulic pressure reading, mix design, job name and truck status. Criteria/wickets for proper alerting include load size, drum rpm range, hydraulic pressure stability and truck status. More details regarding alerting criteria and a state diagram are set forth below.

The SMARThatch system is set up to send alerts via email or text indicating slump is too wet or too dry. By way of example, below is a sample alert, as follows:

External Sender
The Concrete Slump Alert - Threshold rule for Concrete Hydraulic
Pressure/Slump Alert on asset #3672 has detected a value
state change at Jun. 1, 2022, 11:48:28 AM EDT.
View Asset

Truck#: 3672
Signal: Concrete Hydraulic Pressure/Slump Alert
Time/Date: Jun. 1, 2022, 11:48:28 AM EDT
Rule Title: Concrete Hydraulic Pressure/Slump Alert - Threshold
Value Received: 1800 psi
Threshold Limit: 1750 psi
Truck Status: loaded in yard
Job #: 24524
Mix Design: 23524-1

### Specific Embodiments

In its broadest sense, the present invention provides a new and unique apparatus for providing a slump alert featuring a signal processor or processing module configured to:
receive signaling containing information about a hydraulic pressure of a hydraulic fluid used or required to rotate a drum of a concrete truck having concrete contained therein; and
provide corresponding signaling containing information about a slump alert of the concrete contained in the drum, based upon the signaling received.

The apparatus may include one or more of the following features:

The signal processor or processing module may be configured to determine the slump alert based upon a measurement of the hydraulic pressure of the hydraulic fluid used or required to rotate the drum at a specific rotation speed.

The apparatus may include a hydraulic pressure measurement device configured to measure the hydraulic pressure used or required to rotate the drum and provide hydraulic pressure measurement signaling containing information about the hydraulic pressure measured.

The apparatus may include a memory having a look-up table containing information about reference slumps for corresponding reference concretes that are in-specification and out-of-specification based upon the hydraulic pressure measured.

The signal processor or processing module may be configured to index the look-up table based upon the signaling.

The look-up table may contain information about one or more of the following:
a group or type of concrete truck,
a mix design of the concrete,
a size of the concrete load,
a drum rotation speed, and
a hydraulic pressure threshold corresponding to in-specification and out-of-specification of a specification slump.

The signaling also may contain information about one or more of the following:
the speed of rotation of the drum,
the weight/amount of wet concrete in the drum,
a mix design of the wet concrete in the drum,
the type of concrete truck containing the wet concrete, and
a slump consistency of the wet concrete.

The slump alert may include information about one or more of the following:
a truck no.
a signal containing information about a concrete hydraulic pressure/slump alert,
a date and time of the slump alert,
a rule title of the slump alert containing information about a threshold indication of concrete hydraulic pressure/slump alert,
a hydraulic pressure measured,
a threshold limit of the hydraulic pressure,
a hydraulic pressure range and/or stability
a truck status indication containing information about whether the concrete truck is loaded in the yard,
a job name or #,
a concrete load size,
a drum RPM range, and
a mix design of the concrete.

The signal processor or processing module may be configured to send the slump alert via an email or text message, including indicating that the slump of the concrete is too wet or too dry.

The signal processor or processing module may be configured to receive a customer action button containing information that enables a customer to indicate action has been taken after the slump alert was received.

The signal processor or processing module may be configured to receive further signaling containing information about water added to the concrete in the drum, including the number of gallons added, and provide in the slump alert further information about the water added to the concrete in the drum, including the number of gallons added.

The corresponding signaling may contain further information that includes a report to identify trends and/or operational inefficiencies used to reduce slump related problems.

According to some embodiments, the apparatus may take the form of a signal processor or processing module configured to:
receive signaling containing information about a drum motor drive characteristic required to rotate a drum of a concrete truck having concrete contained therein; and
provide corresponding signaling containing information about a slump alert of the concrete contained in the drum, based upon the signaling received.

By way of example, the drum motor drive characteristic may be a measurement of a hydraulic pressure used or required to rotate the drum or a speed of rotation of the drum.

According to some embodiments, the present invention may include, or take the form of, a method comprising steps of:
receiving, with a signal processor or processing module, signaling containing information about a hydraulic pressure of a hydraulic fluid used or required to rotate a drum of a concrete truck having concrete contained therein; and
providing, with the signal processor or processing module, corresponding signaling containing information about a slump alert of the concrete contained in the drum, based upon the signaling received.

The method may also include one or more of the features set forth herein.

### BRIEF DESCRIPTION OF THE DRAWING

The drawing includes FIGS. 1-4b which are not necessarily drawn to scale, as follows:

FIG. 1 shows a block diagram of apparatus according to some embodiments of the present invention.

FIGS. 2A and 2B show a flow diagram having a breakdown of steps for implementing the present invention by providing final computations, according to some embodiments of the present invention.

FIG. 3 is a graph of slump (inches) versus time (e.g., for a given day (MM/DD/YYYY), time (HH:MM:SS AM/PM EST)) that may be displayed over periods that include a ½ H period (as shown), a 3 H period, a 12 H period or live, that shows top and bottom functions, the bottom function being a real-time hydraulic pressure reading (PSI) and the top function being a real time drum (RPMs), that includes and displays a reading R on Jan. 4, 2022 at 3:50:15 AM EST having a drum RPM of 16.5 RPMs, a hydraulic pressure of 1580 PSI and a water volume of 0 Gallons.

FIG. 4A shows a known concrete mixer truck having a cab, a mixer drum capable of carrying a concrete load, a drive assembly for rotating mixer drum and a power system driven by a transmission of the mixer truck.

FIG. 4B shows the power system in FIG. 4A in further detail, which includes the transmission, a power takeoff drive gear, a mechanical drive line, a hydraulic pump, fluid lines between the hydraulic pump and the drive assembly, hydraulic lines between the hydraulic pump and a fluid reservoir.

### DETAILED DESCRIPTION OF BEST MODE OF THE INVENTION

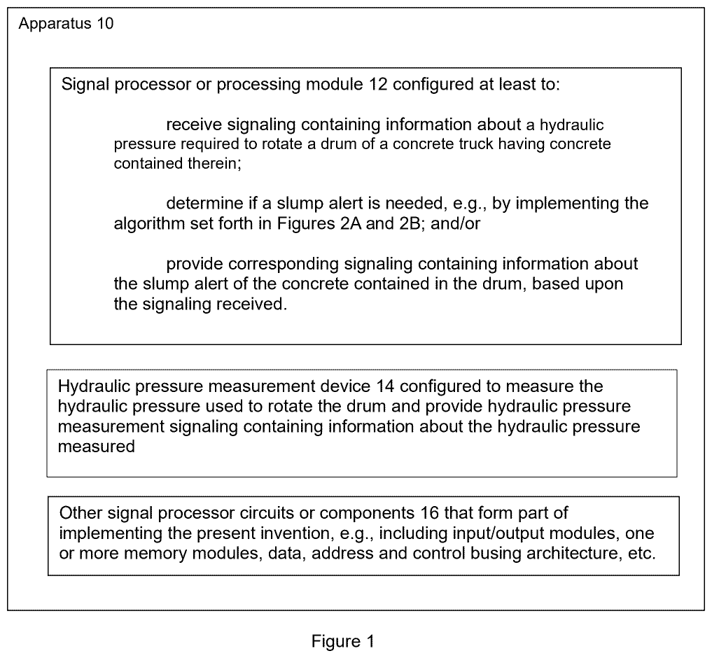

### FIG. 1

By way of example, and consistent with that disclosed herein, including that shown in FIG. 1, the present invention may include, or take the form of, apparatus generally indicated as 10 for providing a slump alert featuring a signal processor or processing module 12 configured to:
receive signaling containing information about a hydraulic pressure of a hydraulic fluid used or required to rotate a drum of a concrete truck having concrete contained therein; and
provide corresponding signaling containing information about a slump alert of the concrete contained in the drum, based upon the signaling received.

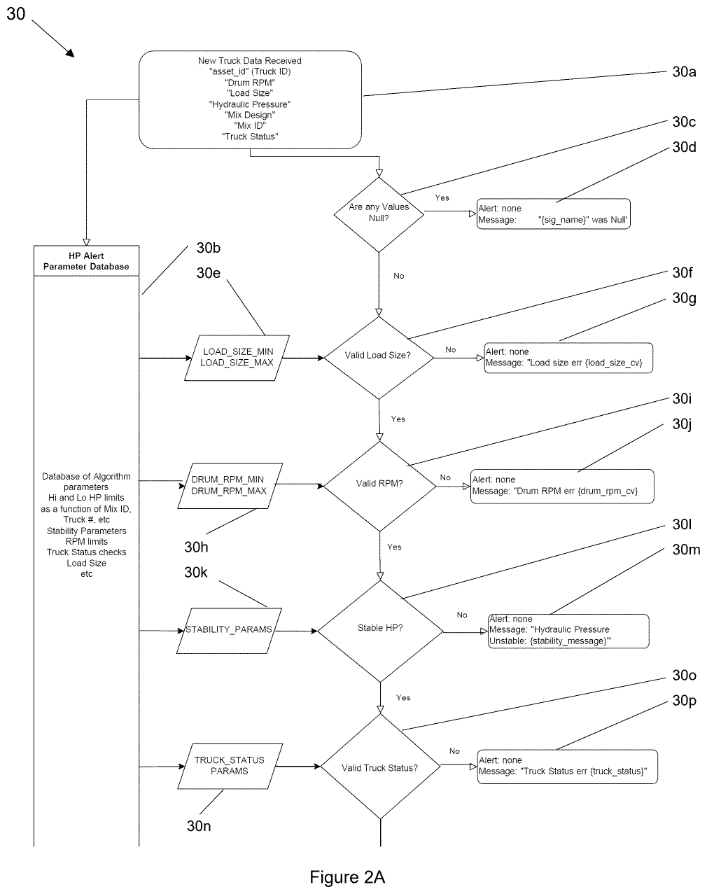

By way of example, the signal processor or processing module 12 may be configured to determine if a slump alert is needed, e.g., by implementing the algorithm set forth in FIGS. 2A and 2B, e.g., consistent with that set forth herein.

The signal processor or processing module 12 may be configured to determine the slump alert based upon a measurement of the hydraulic pressure of the hydraulic fluid used or required to rotate the drum at a specific rotation speed. The apparatus 10 may include a hydraulic pressure measurement device 14 configured to measure the hydraulic pressure of the hydraulic fluid used or required to rotate the drum and provide hydraulic pressure measurement signaling containing information about the hydraulic pressure measured. Hydraulic pressure measurement device like element 14 are known in the art, and the scope of the invention is not intended to be limited to any particular type or kind either now known or later developed in the future.

The apparatus 10 may include a memory 16 having a look-up table containing information about reference slumps for corresponding reference concretes that are in-specification and out-of-specification based upon the hydraulic pressure measured. The signal processor or processing module 10 may be configured to index the look-up table based upon the signaling received. By way of example, the look-up table forming part of the memory 16 may contain information about one or more of the following:
a group or type of concrete truck,
a mix design of the concrete,
a size of the concrete load,
a drum rotation speed, and
a hydraulic pressure threshold corresponding to in-specification and out-of-specification of a specification slump.

By way of example, the signaling also may contain information about one or more of the following:
the speed of rotation of the drum,
the weight/amount of wet concrete in the drum,
a mix design of the wet concrete in the drum,
the type of concrete truck containing the wet concrete, and
a slump consistency of the wet concrete.

The slump alert may include information about one or more of the following:
a truck no.

a signal containing information about a concrete hydraulic pressure/slump alert,
a date and time of the slump alert,
a rule title of the slump alert containing information about a threshold indication of concrete hydraulic pressure/slump alert,
a hydraulic pressure measured,
a threshold limit of the hydraulic pressure,
a hydraulic pressure range and/or stability
a truck status indication containing information about whether the concrete truck is loaded in the yard,
a job name or #,
a concrete load size,
a drum RPM range, and
a mix design of the concrete.

The signal processor or processing module 12 may be configured to send the slump alert via an email or text message, including indicating that the slump of the concrete is too wet or too dry, according to some embodiments of the present invention. In such embodiments, the apparatus may include corresponding email or text message applications for interfacing with associated email or text message applications.

The signal processor or processing module 12 may be configured to receive a customer action button containing information that enables a customer to indicate action has been taken after the slump alert was received. In such embodiments, the apparatus may include corresponding customer interfacing applications to enable the customer to activate the action button, e.g., via a customer app downloaded on a smart phone, or an iPad, or a laptop or desktop computer, etc.

The signal processor or processing module 12 may be configured to receive further signaling containing information about water added to the concrete in the drum, including the number of gallons added, and provide in the slump alert further information about the water added to the concrete in the drum, including the number of gallons added. By way of example, the further signaling may be provided a water provisioning device adding the water the concrete in the drum, or by an operator of such a water provisioning device, e.g., via an app on a smart phone, or an iPad, or a laptop or desktop computer, etc.

The corresponding signaling may contain further information that includes a report to identify trends and/or operational inefficiencies used to reduce slump related problems.

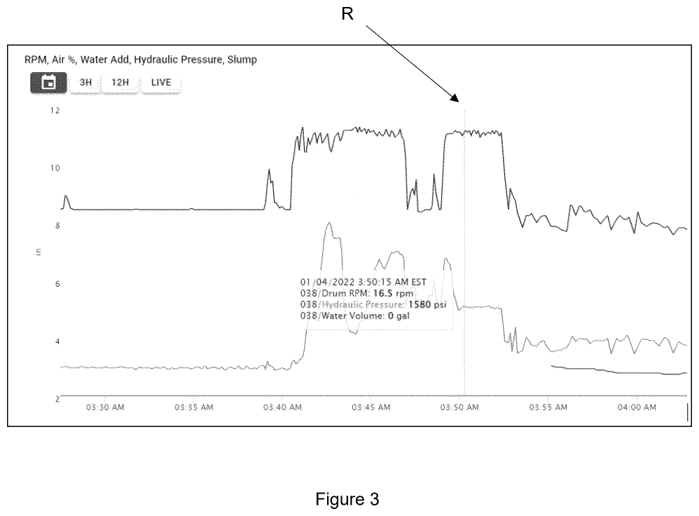

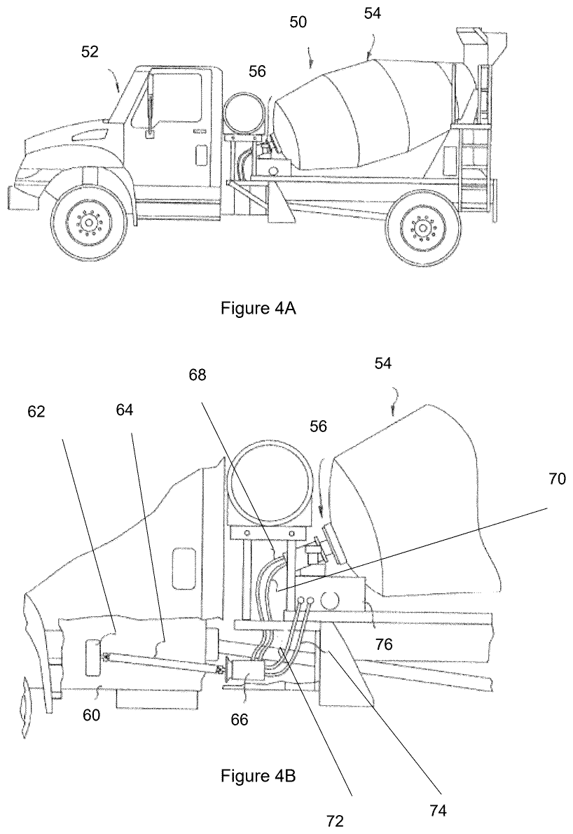

By way of example, the concrete truck may include, or take the form of that shown in FIG. 4A, although the scope of the invention is intended to include other types or kinds of concrete trucks either now known or later developed in the future. For example, see the concrete trucks disclosed in the Assignee's aforementioned technology.

### FIGS. 2A and 2B

By way of example, FIGS. 2A and 2B show a flow diagram of an algorithm generally indicated as 30 for implementing the present invention to determine is a slump alert is needed. The algorithm includes a series of steps 30a and 30c to 30w for the signal processor or processing module 12 to implement and interact with a hydraulic pressure (HP) alert parameter database 30b, which includes a database algorithm parameter (such as Hi and Lo HP limits as a function of a Mix ID, Truck #, etc.; stability parameters, drum RPM min max limits, truck status checks, load size min max limits, etc.).

In step 30a, the signal processor or processing module 12 receives and processes new truck data, e.g., which includes an “Asset ID” (e.g., a Truck ID), “Drum RPM”, “Load size”, “Hydraulic pressure”, “Mix Design”, “Mix ID” and “Truck status”.

After receiving the new truck data, the signal processor or processing module 12 implements the algorithm 30 steps 30c to 30w in order to determine a current criteria that must be met as follows:
Steps 30c and 30d: Truck Data Received must not be null value.
Steps 30e to 30g: A load size is ##yd³ or greater.
Steps 30h to 30j: Drum RPM must be greater than ##RPM and less than ##RPM.
Steps 30k to 30m: Hydraulic pressure must be stable.
   Must maintain a value that has a delta value less than XXX over YYY time.
Steps 30n to 30p: Truck status must be valid.
Steps 30q to 30v: Hydraulic pressure must be within the bounds of previously acquired bump test data, including if HP>Hi HP limit and if HP<Lo HP limit.

When the steps are implemented, the algorithm provides a breakdown and final computations to determine if a slump alert is needed or not (e.g., in step 30w, which indicates that the HP is in a good slump range).

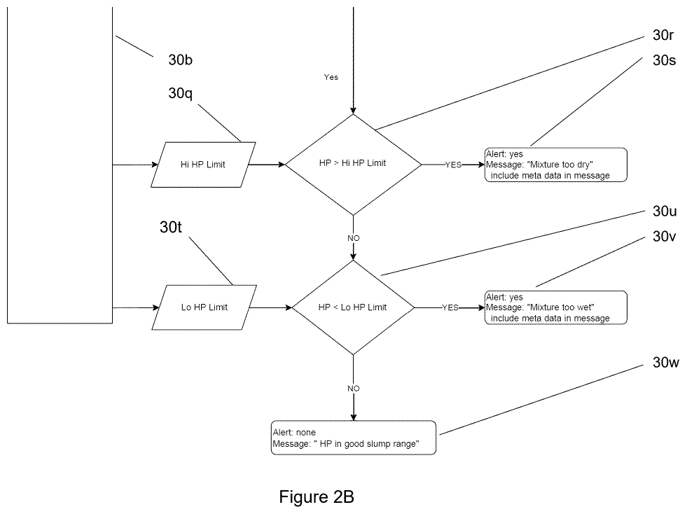

### FIG. 3

FIG. 3 shows a graph of slump (inches) versus time (e.g., for a given day (MM/DD/YYYY), time (HH:MM:SS AM/PM EST)) that may be tracked and/or displayed over periods that include a ½ H period (as shown), a 3 H period, a 12 H period or live, that shows top and bottom functions, the bottom function being a real-time hydraulic pressure reading (PSI) and the top function being a real time drum rotation reading (RPMs), that includes and displays a particular reading R on Jan. 4, 2022 at 3:50:15 AM EST having a drum rotation of 16.5 RPMs, a hydraulic pressure of 1580 PSI and a water volume of 0 Gallons.

### The Signal Processor or Processing Module 12

The functionality of the signal processor or processor control module 12 may be implemented using hardware, software, firmware, or a combination thereof. In a typical software implementation, the processor module may include one or more microprocessor-based architectures having a microprocessor, together with other signal processor circuits or components 16, e.g., including a random access memory (RAM), a read only memory (ROM), input/output devices and control, data and address buses connecting the same, e.g., consistent with that shown in FIG. 1. The apparatus includes other signal processor circuits or components collectively indicated as 16, including a memory or memory module configured to store data/information and a computer program, e.g., including a table lookup and an algorithm having steps for implementing some aspects of the present invention like that shown in FIG. 2, etc.

A person skilled in the art would be able to program such a microprocessor-based architecture(s) to perform and implement such signal processing functionality described herein without undue experimentation. The scope of the invention is not intended to be limited to any particular implementation using any such microprocessor-based architecture or technology either now known or later developed in the future.

### FIGS. 4A and 4B

By way of example, FIG. 4A shows a known concrete mixer truck 50 having a cab 52, a mixer drum 54 capable of carrying a concrete load, a drive assembly 56 for rotating the mixer drum 54 and a power system 58 driven by a transmission 60 of the mixer truck 50, e.g., consistent with that shown in US 2008/0198686, which is hereby incorporated by reference in its entirety.

FIG. 4B shows the power system 58 in FIG. 4A in further detail, which includes the transmission 60, a power takeoff drive gear 62, a mechanical drive line 64, a hydraulic pump 66, fluid lines 68, 70 between the hydraulic pump 66 and the drive assembly 56, hydraulic lines 72, 74 between the hydraulic pump 66 and a fluid reservoir 76, e.g., again consistent with that shown in US 2008/0198686.

In operation, in order to rotate the mixer drum 54, the hydraulic pump 66 provides hydraulic fluid from its discharge port via a fluid feed line 68 to an input port of the drive assembly 56, and receives the hydraulic fluid back from the drive assembly 56 to the hydraulic pump 66 via a fluid return line 70.

By way of example, a hydraulic pressure measurement device like element 14 may be configured in relation to the discharge port of the hydraulic pump 66 and/or the input port of the drive assembly 56 receiving the hydraulic fluid from the fluid feed line 68 coupling the hydraulic pump 66 and the drive assembly 56. One skilled in the art would appreciate and understand how to make such a configuration without undue experimentation.

Moreover, techniques for measuring the speed of rotation (RPMs) of the mixer drum like element 54 are known in the art, and the scope of the invention is not intended to be limited to any particular type or kind thereof either now known or later developed in the future.

### The Scope of the Invention

While the invention has been described with reference to an exemplary embodiment, it will be understood by those skilled in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. Apparatus comprising:
a signal processor or processing module configured to:
receive signaling containing information about a hydraulic pressure of a hydraulic fluid used or required to rotate a drum of a concrete truck having concrete contained therein; and
provide corresponding signaling containing information about a slump alert of the concrete contained in the drum, based upon the signaling received.

2. Apparatus according to claim 1, wherein the signal processor or processing module configured to determine the slump alert based upon a measurement of the hydraulic pressure of the hydraulic fluid used or required to rotate the drum at a specific rotation speed.

3. Apparatus according to claim 2, wherein the apparatus comprises a hydraulic pressure measurement device configured to measure the hydraulic pressure of the hydraulic fluid used or required to rotate the drum and provide hydraulic pressure measurement signaling containing information about the hydraulic pressure measured.

4. Apparatus according to claim 1, wherein the apparatus comprises a memory having a look-up table containing information about reference slumps for corresponding reference concretes that are in-specification and out-of-specification based upon the hydraulic pressure measured.

5. Apparatus according to claim 4, wherein the signal processor or processing module may be configured to index the look-up table based upon the signaling.

6. Apparatus according to claim 4, wherein the look-up table contains information about one or more of the following:
a group or type of concrete truck,
a mix design of the concrete,
a size of the concrete load,
a drum rotation speed, and
a hydraulic pressure threshold corresponding to in-specification and out-of-specification of a specification slump.

7. Apparatus according to claim 1, wherein the signaling also contains information about one or more of the following:
the speed of rotation of the drum,
the weight/amount of wet concrete in the drum,
a mix design of the wet concrete in the drum,
the type of concrete truck containing the wet concrete, and
a slump consistency of the wet concrete.

8. Apparatus according to claim 1, wherein the slump alert includes information about one or more of the following:
a truck no.
a signal containing information about a concrete hydraulic pressure/slump alert,
a date and time of the slump alert,
a rule title of the slump alert containing information about a threshold indication of concrete hydraulic pressure/slump alert,
a hydraulic pressure measured,
a threshold limit of the hydraulic pressure,
a hydraulic pressure range and/or stability
a truck status indication containing information about whether the concrete truck is loaded in the yard,
a job name or #,
a concrete load size,
a drum RPM range, and
a mix design of the concrete.

9. Apparatus according to claim 1, wherein the signal processor or processing module configured to send the slump alert via an email or text message, including indicating that the slump of the concrete is too wet or too dry.

10. Apparatus according to claim 1, wherein the signal processor or processing module configured to receive a customer action button containing information that enables a customer to indicate action has been taken after the slump alert was received.

11. Apparatus according to claim 1, wherein the signal processor or processing module is configured to receive further signaling containing information about water added to the concrete in the drum, including the number of gallons added, and provide in the slump alert further information about the water added to the concrete in the drum, including the number of gallons added.

12. Apparatus according to claim 1, wherein the corresponding signaling contains further information that includes a report to identify trends and/or operational inefficiencies used to reduce slump related problems.

13. Apparatus according to claim 1, wherein the signal processor or processing module is configured to determine a slump alert by indexing the signaling in a memory having a look-up table containing information about reference slumps for corresponding reference concretes that are in-specification and out-of-specification based upon the hydraulic pressure measured

14. Apparatus comprising:
a signal processor or processing module configured to:
receive signaling containing information about a drum motor drive characteristic required to rotate a drum of a concrete truck having concrete contained therein; and
provide corresponding signaling containing information about a slump alert of the concrete contained in the drum, based upon the signaling received.

15. Apparatus according to claim 14, wherein the drum motor drive characteristic is a measurement of a hydraulic pressure used or required to rotate the drum.

16. Apparatus according to claim 14, wherein the drum motor drive characteristic is a measurement of a speed of rotation of the drum.

17. A method comprising:
receiving, with a signal processor or processing module, signaling containing information about a hydraulic pressure of a hydraulic fluid used or required to rotate a drum of a concrete truck having concrete contained therein; and
providing, with the signal processor or processing module, corresponding signaling containing information about a slump alert of the concrete contained in the drum, based upon the signaling received.

18. A method according to claim 17, wherein the method further comprises determining, with the signal processor or processing module, the slump alert based upon a measurement of the hydraulic pressure of the hydraulic fluid used or required to rotate the drum at a specific rotation speed.

19. A method according to claim 18, wherein the method further comprises configuring a hydraulic pressure measurement device to measure the hydraulic pressure used or required to rotate the drum and provide hydraulic pressure measurement signaling containing information about the hydraulic pressure measured.

20. A method according to claim 17, wherein the method further comprises configuring a memory with a look-up table containing information about reference slumps for corresponding reference concretes that are in-specification and out-of-specification based upon the hydraulic pressure measured.
